(19) United States
(12) Patent Application Publication
(10) Pub. No.: US 2009/0062555 A1
(43) Pub. Date: Mar. 5, 2009

(54) Synthesis of Isoflavone

(76) Inventor: Arthur E. Harms, Overland Park, KS (US)

Correspondence Address: Bausch & Lomb Incorporated, One Bausch & Lomb Place, Rochester, NY, 14604-2701, US

(21) Appl. No.: 12/198,137

(22) Filed: Aug. 26, 2008

Related U.S. Application Data
(60) Provisional application No. 60/968,407, filed on Aug. 28, 2007.

(51) Int. Cl. C07D 311/38

(52) U.S. Cl. 549/403

(57) ABSTRACT

The invention provides a process of manufacturing an isoflavone of the general formula 7

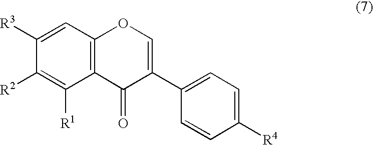

wherein R¹, R², R³, and R⁴ each independently are H, OH, or an alkoxy, provided at least one of R¹, R², R³, and R⁴ being OH, and one being alkoxy, or at least 2 groups being OH, comprising providing ketone (3)

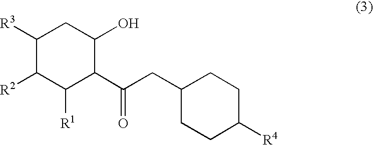

wherein R¹, R², R³, and R⁴ are as stated above, combining (3) with trialkylortho formate, and a Lewis or Bronsted acid to produce the isoflavone, precipitating the isoflavone.

### CROSS-REFERENCE

This application claims the benefit of U.S. Provisional Patent Application No. 60/968,407 filed Aug. 28, 2007, which is incorporated by reference herein.

### BACKGROUND OF THE INVENTION

1. Field of the Invention

The present invention concerns a process for manufacture of certain isoflavones. Of particular interest is the formation of genistein.

2. Discussion of the Prior Art

Isoflavones display many useful biochemical effects. For example, the naturally occurring and commercially available substance genistein has been claimed to be useful as an anti-inflammatory agent, for prevention and treatment of osteoporosis and heart disease, for prevention of photodamage and aging skin and wrinkles, for inhibition of Alzheimers disease and for treatment of menopausal symptoms, estrogen disorders, cancer, cataracts, cystic fibrosis and migraine. The synthetic and commercially available isoflavone irpiflavone has been claimed to be useful for treatment of osteoporosis and estrogen disorders. Amongst the literature in this field, M. Messina, Chemistry & Industry 1995, 413-415, and T. E. Wiese et al., ibid. 1997, 648-653, present interesting reviews on the biological effects and uses of isoflavones, including genistein.

The naturally occurring isoflavones formononetin and biochanin A, which in contrast to genistein and daidzein do not occur in soy, are of agricultural interest as promoters for mycorrhizal fungi which benefit plant growth.

Isoflavones are characterized by the following general benzopyranone structure, the ring numbering also being shown:

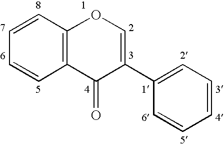

Many substances containing the isoflavone structure are found naturally, mainly in the family Leguminosiae; the richest sources are soy, lentils, chickpea, fenugreek, clover, alfalfa and various types of beans. Genistein, for example, was first isolated in 1899 from broom (Genista tinctoria), and later, in 1931, isolated from Soya hispida. See A. G. Perkins et al., J. Chem. Soc., Vol. 75, 830 (1899), and E. Walz, Ann. Chem., Vol. 489, 118 (1931). Its structure was first determined by W. Baker and R. Robinson in 1926 (J. Chem. Soc. 1926, 2713), and the compound later synthesized (ibid. 1928, 3115).

Most frequently, the isoflavones are substituted on the rings to varying degrees with hydroxyl, alkoxy, aryl (e.g., phenyl and phenyl-derived) groups, and the molecule may constitute part of a more complex ring structure. Naturally occurring isoflavones are often substituted at the hydroxy groups with sugars, which on their part are sometimes additionally substituted by ester groups. Isoflavone are commonly isolated in nature as mixtures with closely related substances. For example, isoflavones isolated from soybeans include genistein and daidzein which occur co-mixed as free aglycones (sugar-free forms) as well as their glycosides genistin and daidzin in soybeans to the extent of about 500-3000 ppm on a dry weight basis. Direct isolation from biomass containing such mixtures is thus complex and often largely economically impracticable.

World Patent WO 2004/009576—Burdet et al. discloses manufacturing a hydroxylated isoflavone by reacting a 2-hydroxydeoxybenzoin with a formic anhydride in the presence of the base or a solvent which acts as a base.

World Patent WO 2/085881—Burdick et al. discloses a process for the preparation of isoflavones. The process involves the reaction of a precursor with 2-hydroxyaryl alkyl ketone in the presence of a formic-sulfuric anhydride salt.

U.S. Pat. No. 4,264,509—Zilliken discloses a process for production of isoflavones using tempeh as a starting material to produce antioxidant materials. The antioxidant materials and may be further modified to produce the isoflavones.

U.S. Pat. No. 5,247,102—Kallay et al discloses the preparation of substituted isoflavone derivatives. The materials are formed by reaction of a resorcinol derivative with ethyl orthoformate.

There is a need for an improved process capable of providing on manufacturing scale isoflavones with a high degree of purity and without the presence of it materials that would be harmful to living mammals.

### BRIEF SUMMARY OF THE INVENTION

The invention provides a process of manufacturing an isoflavone of the general formula

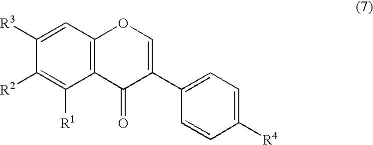

wherein R¹, R², R³, and R⁴ groups each independently are H, OH, or a C₁-C₁₀ alkoxy, provided at least one of R¹, R², R³, and R⁴ groups is OH and another one of R¹, R², R³, and R⁴ groups is alkoxy, or at least 2 groups being OH, comprising providing ketone (3)

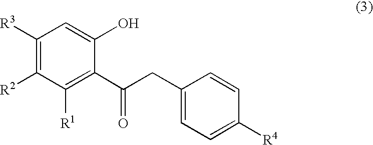

wherein R¹, R², R³, and R⁴ are as stated above, combining (3) with trialkylortho formate, and a Lewis or Bronsted acid, precipitating the isoflavone and recovering the isoflavone after precipitation.

### DETAILED DESCRIPTION OF THE INVENTION

The invention has with many advantages over the prior processes for forming isoflavones particularly genistein. The process results in very pure product without impurities harmful to living animals. The process utilizes relatively low-cost materials and processes to achieve the formation of useful isoflavones.

In a preferred form the invention provides a process of manufacturing an isoflavone of the general formula 7

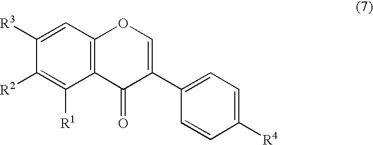

wherein R¹, R², R³, and R⁴ each independently are H, OH, or a C₁-C₁₀ alkoxy, provided at least one being OH, and one being alkoxy or at least 2 groups being OH. In a preferred form, the alkoxy comprises C₁-C₃; a preferred process comprises combining the phloroglucinol of compound (1)

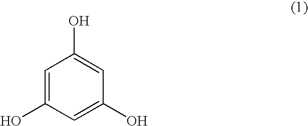

with the compound (2)

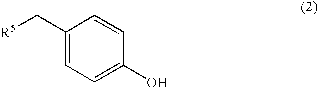

wherein R⁵ comprises nitrile or an ester to produce a ketone (3)

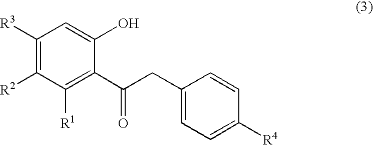

wherein R¹, R², R³, and R⁴ are as stated above, combining (3) with trialkyl orthoformate, and a Lewis or Bronsted acid, precipitating the isoflavone, and recovering the isoflavone after precipitation.

The ketone conversion process of the invention preferably forms the isoflavone

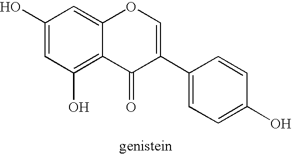

The process of the invention also can produce the isoflavones

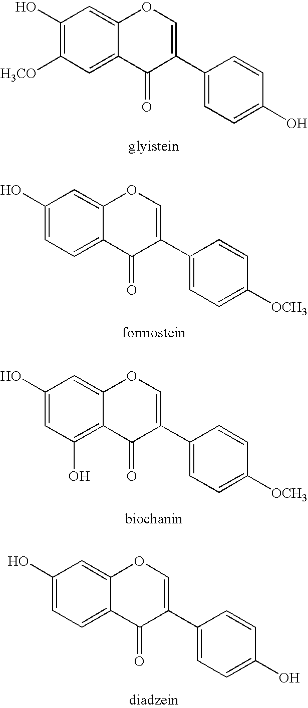

The compound (2) material may comprise an ester of the formula 8.

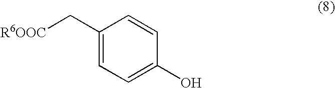

wherein R⁶ comprises alkyl, phenyl, aryl, or benzyl.

Compound (2) in a preferred form comprises the nitrite

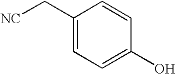

The preferred ketone (9) of compound 3 group is

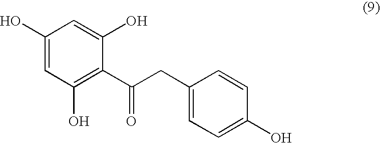

as this material converts to the preferred genistein product efficiently in the presence of (CH₃O)₃ CH and a Lewis or Bronsted acid. The ketone of structure 8 may also be produced by other means than above shown and then converted to the isoflavone genistein by the invention use of (CH₃O)₃ CH and a Lewis or Bronsted acid. Other suitable ways of forming the ketone are shown in the Examples of WO 2002/009576 incorporated herein by reference. The Examples of WO2004/009576 also produce suitable ketones for forming isoflavones by the invention process.

Any suitable trialkylortho formate may be used in the invention process. A suitable material is triethyl orthoformate. A preferred compound is (CH₃O)₃ CH as its use results in effective high yield formation of genistein.

Any suitable Lewis acid or Bronsted acid may be used in the invention. Suitable Lewis acids are aluminum chloride or titanium chloride. A suitable Bronsted acid is sulfuric acid. The preferred materials result in a high yield of isoflavones.

The reactions of the invention are preferably driven to conclusion by combining the starting ingredients compounds 1 and 2 with the trialkyl orthoformate, and Lewis Acid or Bronsted Acid in a suitable solvent.

The ketones as described above are useful precursors for the synthesis of isoflavones which are naturally occurring, as well as synthetic ones.

The process of the present invention may be used to prepare isoflavones with various substituent patterns. The more preferred process involves the production of 5,7 hydroxy isoflavones from 2,4,6-trihydroxyaryl alkyl ketones. These isoflavones are difficult to obtain on large scale by other procedures known in the art. Most preferred is a process for obtaining pure genistein.

Reaction times of 30 minutes to 6 hours are typical with the present invention. Acceptable condensation temperatures of the present process are in the range of from −20° C. to +20° C. It is a desirable feature of this invention that the formylsulfate salts are substantially stable in the reaction media and may be held cold without significant loss of performance. When heated they undergo loss of carbon monoxide in a smooth, non-accelerating manner. Most preferred is an initial reaction temperature in the range of from −10° C. to +10° C. Under these conditions, reactions are completed within less than one to several hours but may be prolonged without loss of performance.

It is a desirable feature of this process that excess of formylating agent may safely be eliminated prior to workup by a brief heating. For this purpose heating of the reaction mixture for 30 minutes to 1 hour is useful, at temperatures which are in the range of from 40° C. to 100° C. During the heating, other phenolic hydroxyl groups which may have been formylated are also regenerated, eliminating a hydrolytic step and reducing formate waste. The evolved carbon monoxide may be burned to relatively environmentally acceptable carbon dioxide.

Thus, the present invention is related to a process as defined above, wherein the process is performed at a temperature in the range of from −20° C. to +20° C., with an optionally heating up of 100° C. The temperature suitably may be in the range of from about below room temperature to about 80° C. Preferably the temperature is between about 20° C. and 60° C.

Solvents are selected to not react with the reagents and to maximize the solubility of the reactants. Complete solution of the reagents is not always necessary. Examples of such solvents are halocarbons, alcohols, ethers, esters, amides, and sulfones. Preferred are more polar solvents such as ethers, esters, and amides. When the bases and salts are amines, less polar solvents are useful. Most preferred solvents are amides such as dimethylformamide and N-methyl pyrrolidinone.

After conversion of the ketone to isoflavone has been accomplished, it is useful to first add to the reaction mixture sufficient acid to neutralize excess base prior to isolation. For this purpose sulfuric acid or bisulfate salts may be used in order to simplify the waste stream. The isoflavone may be isolated by separating it from the salts in a manner generally known to those skilled in the art, such as filtration or extraction, followed by crystallization.

Suitable organic solvents are, in general, polar or slightly polar aprotic solvents. Such solvents are, for example, aliphatic and cyclic ethers, e.g. diethyl ether, diisopropyl ether, dibutyl ether, tert butyl methyl ether, diethylene glycol dimethyl ether, tetrahydrofuran and dioxane; lower aliphatic nitriles, e.g. acetonitrile and propionitrile; lower aliphatic esters, e.g. lower alkyl formates and acetates; dimethylsulphoxide; halogenated, particularly chlorinated, lower aliphatic hydrocarbons, e.g. methylene chloride; aromatic hydrocarbons, e.g. benzene, toluene and xylenes; and lower aliphatic ketones, e.g. acetone, 2-butanone, diethyl ketone, methyl isobutyl ketone, di(lower alkyl)formamides, e.g. dimethylformamide, dimethylacetamide, tetramethylurea and N-methyl-pyrrolidone. The preferred solvent is one which has a boiling point (at atmospheric pressure) of less than 80° C. and which is miscible with ethanol and preferably also with water. In these circumstances, such solvent can, at the termination of the reaction, be replaced with ethanol or aqueous ethanol, and the hydroxylated isoflavone product of the formula I can be isolated through crystallization introduced by the addition of water (in general isoflavones are very sparingly soluble in water).

Moreover, the reaction is conveniently effected at normal pressure, the pressure in general not being critical. Furthermore, the reaction mixture is suitably agitated, in particular stirred, to promote good admixture of the components and ensuing efficient reaction.

In general, the course of the reaction can be observed by such conventional analytical techniques as HPLC, e.g. by monitoring the consumption of the starting 2-hydroxydeoxybenzoin.

To isolate the desired product, directly from the mixture on completion of the reaction to form a formula 7 compound or following the subsequent acid-catalysed hydrolysis for deacylation, this compound is conveniently crystallized out by addition of water, suitably at elevated temperature, e.g. at about 50-60° C. The volume of added water is conveniently about a fifth to about a half of the volume of the mixture containing the product before water addition. Optionally after also cooling the aqueous crystalline medium, e.g. to a temperature from about room temperature to about 0° C., the crystalline product is removed, e.g. by filtration, and if desired can be washed, e.g. with a mixture of ethanol and water, or otherwise purified by conventional methods, for example employing an organic solvent in which the product is at the most sparingly soluble, and submitting the product to one or more recrystallizations. Especially suitable organic solvents for such purposes are ethanol, acetone, mixtures of both or mixtures of each with a relatively small proportion of water. The final step in such a purification procedure is usually a thorough drying of the product at elevated temperature and reduced pressure.

The following example illustrates a non-limiting embodiment of the process of the invention. Variations of materials and process conditions within suitable ranges are also possible in practicing the process of the invention to produce the particular desired isoflavone, as will be recognized by people having ordinary skill in the art. Parts and percentages are by weight unless otherwise indicated.

### EXAMPLE

The apparatus consists of a 250 ml double-walled reactor fitted with a stirrer, a dropping funnel, a distillation column, argon gas purging means, a thermometer and a thermostat.

A solution of the compound 8 ketone in dimethylformamide is provided. To this solution is then added a one molar amount of sulfuric acid (the Bronsted acid), and about a 1 molar amount of the dimethylformate to form the genistein after stirring for 1 hour. The genistein is separated by adding water to precipitate the genistein, separating by filtration and then drying.

Generally, the stirring is carried out along any temperature below the boiling point of the reaction mixture. The temperature may be in the range from about below room temperature to about 80° C. Preferably, the temperature is from about room temperature to about 60° C.

The reaction of the ketone (8) of the Example may be shown as

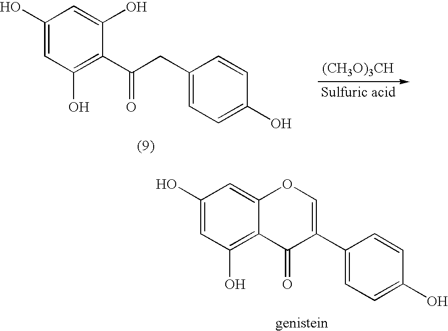

As will be obvious to one skilled in the art, many modifications, variations, substitutions and other alterations can be made in the practices of this invention without departing from the spirit and scope thereof as set forth in the preceding description and examples or in the claims which follow.

What is claimed is:

1. A process of manufacturing an isoflavone of the general formula 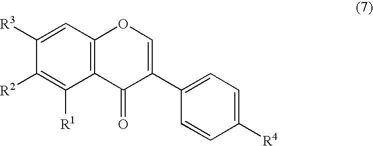
wherein R¹, R², R³, and R⁴ each independently are H, OH, or a C₁-C₁₀ alkoxy, provided at least one of R¹, R², R³, and R⁴ goups is OH, and another of R¹, R², R³, and R⁴ groups is alkoxy or at least 2 groups being OH, said process comprising: (a) providing a ketone having formula 3 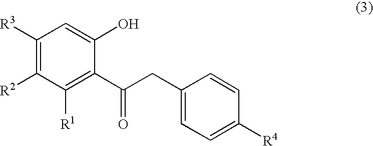
wherein R¹, R², R³, and R⁴ groups are as stated above; (b) reacting said ketone with trialkylortho formate, and a Lewis or Bronsted acid to produce said isoflavone; and (c) precipitating said isoflavone.

2. The process of claim 1, wherein the isoflavone formed comprises

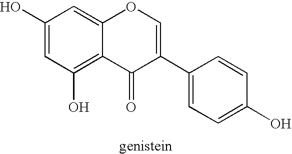

3. The process of claim 1, wherein the isoflavone formed comprises

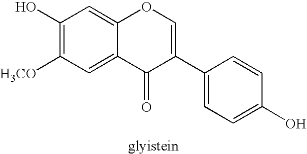

4. The process of claim 1, wherein the isoflavone formed comprises

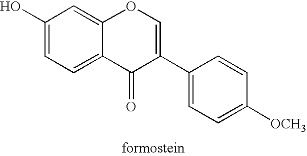

5. The process of claim 1, wherein the isoflavone formed comprises

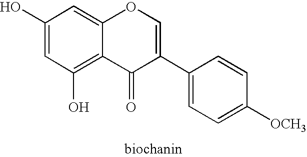

6. The process of claim 1, wherein the isoflavone formed comprises

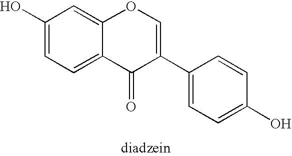

7. The method of claim 1, wherein formation of said ketone having formula 3 is carried out by combining phloroglucinol having formula 1 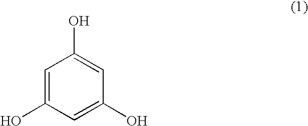
with a compound having formula 2 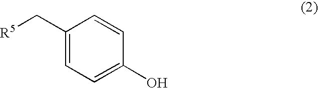
wherein R⁵ comprises nitrile or an ester to produce said ketone.

8. The process of claim 7, wherein the compound having formula 2 comprises 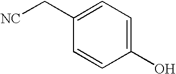
and the ketone comprises

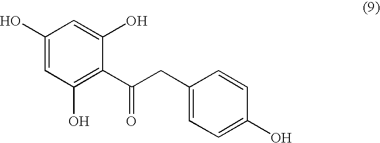

9. The process of claim 1, wherein a Lewis acid is used in the process and the Lewis acid comprises aluminum chloride or titanium chloride.

10. The process of claim 1, wherein a Bronsted acid is used in the process and the Bronsted acid comprises sulfuric acid.

11. The process of claim 1, wherein the trialkylortho formate comprises (CH₃O)₃ CH.

12. The process of claim 1, wherein precipitating as carried out by addition of water.

13. The process of claim 12, further comprising recovering the isoflavone after precipitation.

14. The process of claim 1, wherein the alkoxy group in the compound having 7 is a C₁-C₃ alkoxy.

15. The process of claim 2, wherein after the isoflavone is precipitated it is recovered by filtering and drying.

16. The process of claim 1, wherein the trialkyl orthoforamate comprises triethyl orthoformate.

17. The process of claim 1, wherein the isoflavone formed by the process is a 5,7 hydroxyisoflavone.

18. The process of claim 1, wherein the reacting is carried out in dimethylformamide.

19. The process of claim 1, wherein reacting is carried out in acetone.

20. The process of claim 1, wherein precipitation is carried out in ethanol.
